(19) United States
(12) Patent Application Publication
(10) Pub. No.: US 2010/0112695 A1
(43) Pub. Date: May 6, 2010

(54) Apparatus And Methods For Processing Tissue to Release Cells

(76) Inventor: Kyungyoon Min, Kildeer, IL (US)

Correspondence Address: BAXTER HEALTHCARE CORPORATION, ONE BAXTER PARKWAY, DF2-2E, DEERFIELD, IL, 60015, US

(21) Appl. No.: 12/263,984

(22) Filed: Nov. 3, 2008

(51) Int. Cl. C12N 5/06; C12M 1/00

(52) U.S. Cl. 435/381; 435/308.1; 435/378

(57) ABSTRACT

An apparatus and methods for processing tissue to release biological material including cells are disclosed.

### TECHNICAL FIELD

The present subject matter generally relates to an apparatus and methods for processing tissue to obtain cells.

### BACKGROUND

Biological material often is used in therapeutic, diagnostic or research applications. However, it may be preferable that the material be separated from the tissue from which it derives before being used in these applications. For example, stem cells may originate from several types of tissue including adipose tissue, muscle and blood. It may be desirable to separate the stem cells from the tissue(s) before further processing for introduction into patients or for use in other applications.

To separate biological material from tissue, the tissue often is subjected to a disaggregation or disassociation process. The tissue disaggregation process may involve mechanical means such as homogenization and sonication. In many instances, it may also involve the use of reagents such as enzymes that digest, dissolve or alter the structure of the tissue to effect release of a desired material. For example, to obtain stem cells from an adipose tissue, a solution of an enzyme such as collagenase may be added to digest the connective tissue component of the adipose tissue, thereby releasing the desired stem cells. The use of enzymes such as collagenase may require the control of temperature, pH and other variables during the tissue disaggregation process.

After or even during disaggregation of tissue, the desired material may be subjected to various purification steps, possibly including filtration, centrifugation and affinity methods. There remains a need for an apparatus and methods for processing tissue, including disaggregating and purifying steps, to obtain biological material, including cells.

### SUMMARY

In one example, the disclosure is directed to an apparatus for processing tissue to release cells from the tissue. The apparatus includes a first housing having an outer wall that has a selected shape. The first housing is adapted to receive a tissue sample. The outer and inner walls of the first housing is sufficiently porous to allow passage therethrough of material including cells derived from the tissue. In this example, the apparatus also includes a second housing that at least substantially encloses the first housing and has an outer wall having substantially the same shape as the selected shape of the outer wall of the first housing. At least one of the first and second housings is movable to assist in processing of tissue in the first housing and passage of material including cells derived from the tissue through the porous outer wall of the first housing.

In another example, the disclosure is directed to apparatus for processing tissue to release cells from the tissue where the apparatus has a first housing having an outer wall that has a selected shape. The first housing is adapted to receive a tissue sample and the outer wall is sufficiently porous to allow passage therethrough of material including cells derived from the tissue. The apparatus also includes a second housing that at least substantially encloses the first housing and has an outer wall having substantially the same shape as the selected shape of the outer wall of the first housing. The first and second housings further are disposed at an angle of less than 90° relative to a horizontal plane and the first housing is movable relative to the second housing to assist in moving a fluid over the tissue in the first housing and passing material including cells derived from the tissue through the porous outer wall of the first housing.

The disclosure also is directed to methods for processing tissue. In one example, tissue processing may include releasing cells from tissue. In this example, a tissue sample containing cells is inserted into a first housing. The first housing has an outer wall having a selected shape and being sufficiently porous to allow passage therethrough of material including cells derived from the tissue sample. The first housing is at least substantially enclosed by a second housing having an outer wall that has a shape that is substantially similar to the selected shape of the outer wall of the first housing. The processing further includes introducing tissue-releasing agents into one of the housings. The processing also includes moving at least one of the first and second housings to process the tissue sample and to pass material including cells derived from the tissue sample through the porous outer wall of the first housing.

### BRIEF DESCRIPTION OF THE DRAWINGS

FIG. 1 is a cross-sectional diagrammatic view of an example apparatus for processing tissue;

FIG. 2a is a perspective view of an example apparatus for processing tissue;

FIG. 2b is a partial cross-sectional perspective view of the example shown in FIG. 2a;

FIG. 2c is an exploded view of the apparatus of FIG. 2a;

FIG. 3a is a perspective view of another example apparatus for processing tissue;

FIG. 3b is a partial cross-sectional perspective view of the example shown in FIG. 3a;

FIG. 3c is an exploded view of the apparatus shown in FIG. 3a;

FIG. 4a is a perspective view of a further example of an apparatus for processing tissue;

FIG. 4b is an exploded view of the apparatus shown in FIG. 4a;

FIG. 5 is a partial cross-sectional view of the apparatus for processing tissue of FIG. 4a;

FIG. 6 is a schematic flow chart of exemplary steps for processing tissue.

FIG. 7 is a cross-sectional diagrammatic view of a further example of an apparatus for processing tissue employing an agitator.

### DETAILED DESCRIPTION

While detailed examples are disclosed herein, it is to be understood that these disclosed examples are merely exemplary, and various aspects and features described herein may have utility alone or in combination with other features or aspects in a manner other than explicitly shown but would be apparent to a person of ordinary skill in the art.

The subject matter of this application is directed generally to an apparatus and method for processing tissue to obtain biological material. In a preferred example, the apparatus is used to process adipose tissue to release cells, particularly stem cells.

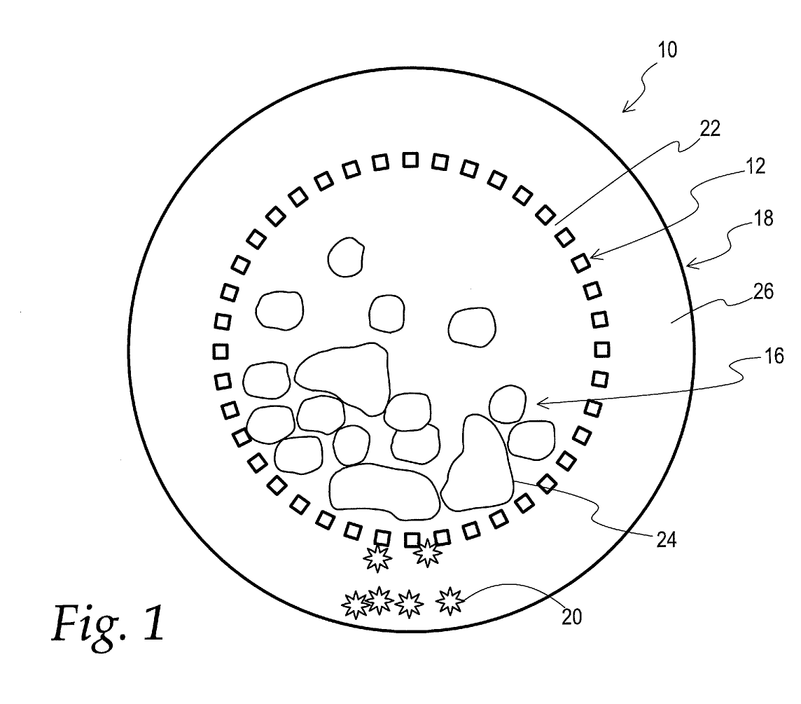

In accordance with this description, an apparatus for processing tissue is shown in a cross-sectional diagrammatic view generally at 10 in FIG. 1. The apparatus includes a first housing 12 having an outer wall 22. The first housing 12 is adapted to receive a tissue sample 16. The apparatus 10 also includes a second housing 18, sized such that the first housing 12 is substantially located within or enclosed by the second housing 18.

At least a portion of the outer wall 22 of the first housing 12 is porous. The porous portion of the outer wall 22 of the first housing 12 allows desired material to pass therethrough while other, undesired, material is retained in the first housing. For example, cells 20 may be released from the tissue sample 16 during a disaggregation procedure and may pass from the first housing 12 through the pores of the outer wall 22 of the first housing 12, while larger tissue fragments 24 may be retained in the first housing 12. The cells 20 that pass through the porous portion of the outer wall of the first housing 12 may pass initially into a space 26 between the first and second housings 12,18.

In one example, the first and second housings 12,18 have substantially the same shape with respect to the selected shape of their respective outer wall. The housings may be, for example, substantially cylindrical. In one example, the diameters of the first and second housings may be selected such that the second housing closely circumscribes the first housing, leaving only a small space 26 defined between the walls of the first and second housings.

The first and second housings 12, 18 may be formed from one or more of a variety of materials, including disposable materials. In a preferred example, the housings also are formed from materials in a manner to make the housings substantially rigid. The materials may include glass, plastic, and metal. In one example, the second housing may be composed, at least in part, of a relatively transparent material that allows the space enclosed by the second housing, including the first housing, to be visualized.

In an example apparatus, the porous portion of the outer wall 22 of the first housing 12 may be formed from a mesh panel. The mesh panel may include a molded sheet having apertures, a non-woven membrane or a web or net structure having strands of one or more materials are woven together to form a porous structure. Materials useful in this apparatus may be of the type described in U.S. Pat. Nos. 6,491,819; 5,194,145; 6,497,821, or in U.S. Published Application 20050263452, all incorporated by reference herein. The materials of the mesh panel may be coated with materials that prevent tissue, cells or molecules or reagents from adhering to the wall or from chemically reacting with the wall. The porous portion, for example, may include metal wire woven together and coated with Teflon. The pores of the outer wall 22 may be sized so as to be the equivalent of being in range from about 5 μm to about 3000 μm in diameter. In a preferred example, the pores are about 200 μm in diameter. Additionally, the outer wall 22 of the first housing 12 may be modified such that tissue processing or purification agents are bound to or incorporated into the wall materials.

In various examples, at least one of the first and second housings 12, 18 may be movable to assist in the processing of tissue and the passage of material such as cells through the porous outer wall 22 of the first housing 12. The housings may be shaken, rotated, agitated or otherwise moved, as desired. The movement of one or both housings may, for example, prevent tissue fragments 24 from adhering to the first housing 12 and may also facilitate the even distribution of the tissue-releasing agent(s) throughout the tissue sample. In one example, the first housing is rotated relative to the second housing. The rotation speed may be, for example, one revolution per second. However, it will be appreciated that other speeds may be chosen as desired. Such rotating action may be used to increase the shear rate between porous outer wall and the liquid portion to prevent plugging of the porous outer wall by the solid portion of the material. Thus, the rotating speed can be varied to achieve a desired shear rate at the surface of the porous outer wall.

In some examples, movement of the housings may be accomplished by fitting the housings into a durable or reusable device with an underlying base which may include devices such as one or more motors which are adapted to interact with and move the housings. The base may also include devices to control and monitor the temperature, pH and other variables.

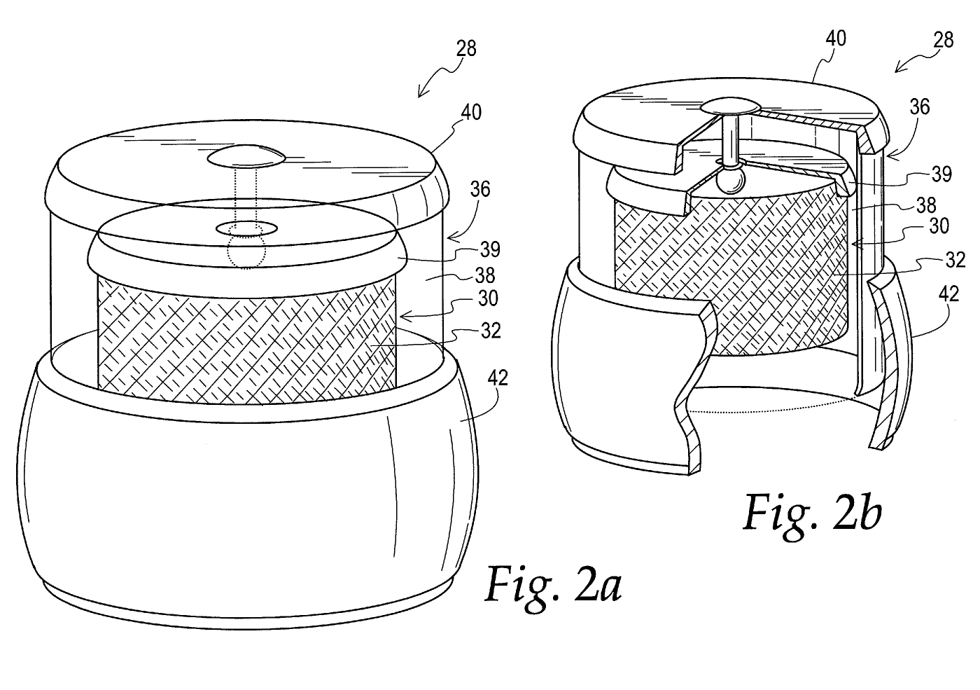

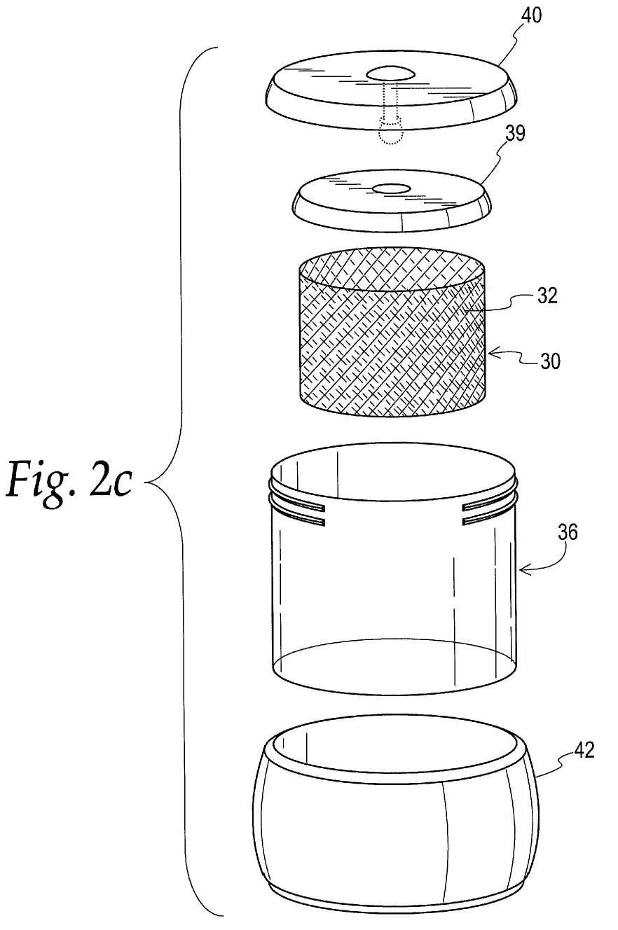

Turning now to FIGS. 2a-2c, an example of a tissue processing apparatus is shown in three views. The apparatus 28 includes a first housing 30 that includes a porous outer wall 32. Although the outer wall 32 of the first housing 30 is shown as being almost entirely porous in this example, the wall may be porous only in part, as desired. Also, in this example, the outer wall 32 of the first housing 30 is substantially cylindrical. The pore size of the wall 32 is selected to allow passage of desired biological material, such as cells derived from the tissue that is placed in the first housing 30. As shown in this example, the first housing 30 is enclosed by a second housing 36. There may be a space 38 defined between the first and second housings 30, 36. In addition, the first and second housings may be removable to facilitate processing, cleaning, or for other purposes.

In the example shown in FIGS. 2a-2c, the first and second housings 30, 36 may have lids or covers 39, 40, that fit an upper opening of the respective housings. The lids may seal the contents of the apparatus 28 from the external environment. The lids or covers 39, 40 may be removable to facilitate placement or removal of tissue, or to otherwise allow access to the contents of the housings when desired. The bottom of each housing also may contain a lid or cover (not shown) or the wall of each housing may be extended to form a bottom wall or surface.

As noted above, the first and second housings may be adapted to fit into a base structure 42. The base 42 may contain a motor for shaking, rotating or otherwise moving the first housing 30 relative to the second housing 36 to agitate at least one of the housings and facilitate tissue disaggregation, and the release of cells from a tissue sample. The base structure also may include devices to control and monitor temperature, pH or other suitable variables. The housings and associated base may employ the principles and structures illustrated in U.S. Pat. No. 5,194,145 in which relative rotation between inner and outer housings creates shear stress to relieve plugging within the device for enhanced filtration.

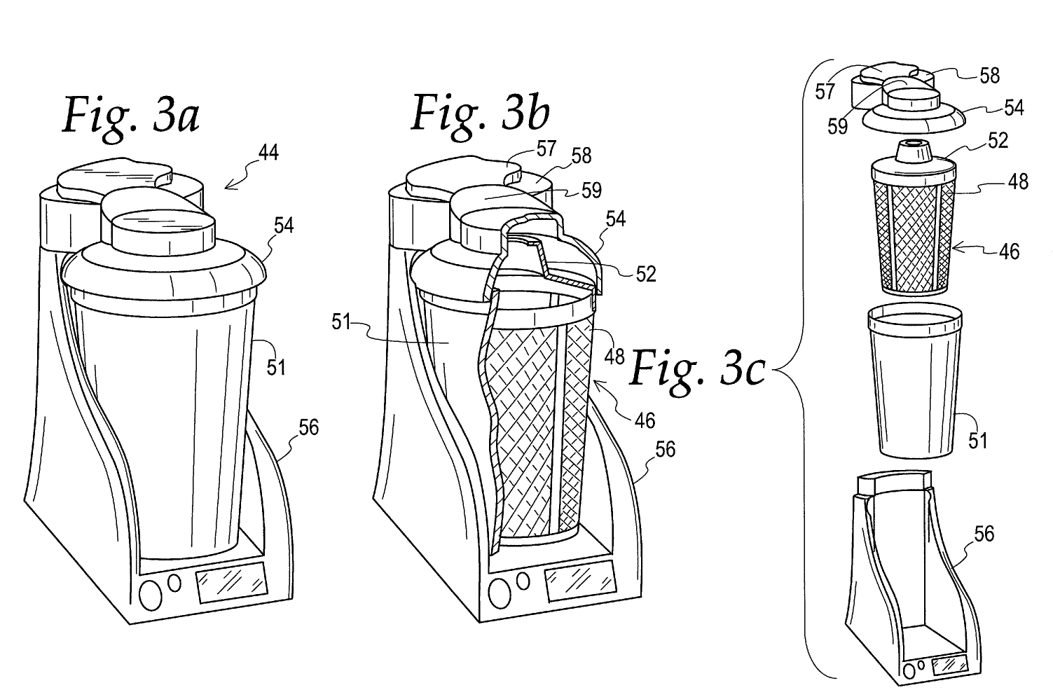

FIGS. 3a-3c represent a further example of an apparatus 44 according to the disclosure. As with the previous examples, a first housing 46 has an outer wall 48 that is adapted to receive a tissue sample. The outer wall 48 is sufficiently porous to allow passage therethrough of material, including cells, derived from the tissue sample and preferably retain undesired material. The first housing 46 is enclosed by a second housing 51. The first and second housings may have lids or covers 52, 54, respectively, and similarly shaped outer walls. A base structure 56 may include devices to rotate the first housing or agitate at least one of the housings and also may include monitors and related systems to detect and control temperature, pH and other variables, as desired. In this example, the base 56 includes a motor, such as a gear or magnetic drive (not shown) which is adapted to drive a cooperative gear or magnetic coupling 57 on a base cover 58, to cause rotation of the first housing 46.

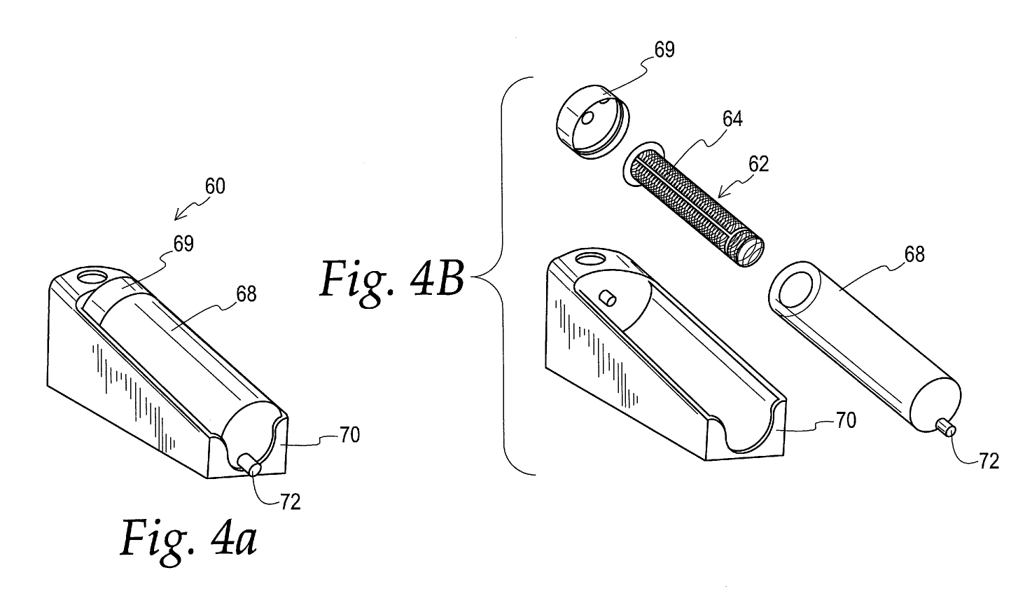

FIGS. 4a and 4b show another example of an apparatus 60 according to the disclosure. In this example, a first housing 62 includes an outer wall 64 and is adapted to receive a tissue sample. The outer wall 64 is sufficiently porous to allow passage therethrough of material including cells derived from the tissue. The first housing 62 is enclosed by a second housing 68 with a cover 69. In this example, the first and second housings 62,68 are positioned in a base 70 at an angle Less than 90° to the surface on which the apparatus 60 rests. This angled or reclined positioning increases the surface area of the tissue within the first housing that may be exposed to a fluid or solution placed in the apparatus 60. In this way, less solution may be used while making contact with more of the tissue in the first housing.

The base 70 may include a motor that may be used to rotate the first housing 62 relative to the second housing 68 to enhance processing of the tissue sample and passage of material, including cells, through the porous wall 64. The base 70 also may include devices to control and monitor temperature, pH and other variables, as desired. In addition, a port 72 may be present in the bottom of the second housing 68 to allow the flow of fluids, including fluids containing biological material such as cells, from the apparatus.

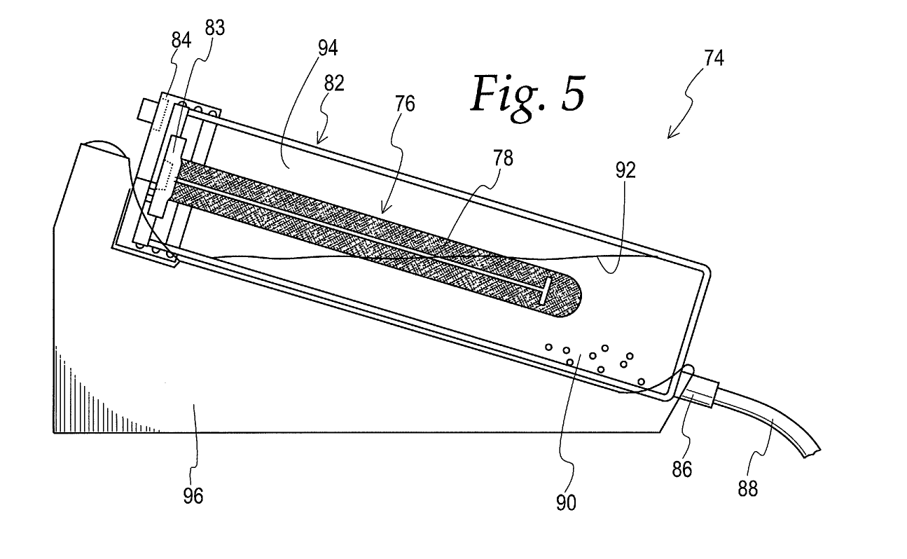

FIG. 5 shows a further example of an apparatus 74 for processing tissue. The cross-sectional view includes released cells 90 and a solution 92, such as a solution of a disaggregation agent. As in previous examples, a first housing 76 includes a porous outer wall 78 and is adapted to receive a tissue sample. The first housing 76 is enclosed by a second housing 82. The first and second housings may have lids or covers 83, 84, respectively, and may be positioned in a base 96. As in previous examples, the base 96 may include one or more motors or drive units such as magnetically or gear coupled drives that may be used to move at least one of the housings, such as to rotate the first housing relative to the second housing, to enhance processing of tissue in the first housing and passage therethrough of material, including cells, derived from the tissue sample. The base 96 also may include devices to control and monitor temperature, pH and other variables, as desired. In addition, an outlet 86 and tubing 88 may be provided so that the biological material, such as cells 90 released during tissue disaggregation, may be flowed out of the second housing 82 of the tissue processor 74.

In accordance with the description and referring generally to FIG. 5, a method of using an apparatus 74 generally includes inserting a tissue sample containing cells (e.g. adipose tissue containing stem cells) into the first housing 76. The tissue sample is subjected to a disaggregation process while placed in the first housing. The disaggregation process may include adding a solution 92 to facilitate release of biological material. Biological material, such as cells 90, may be released during disaggregation and the cells 90 may flow from the first housing 76, through the porous outer wall 78 of the first housing 76. In this example, cells are shown as initially collecting in the space 94 between the first and second housings 76, 82. During the disaggregation procedure, at least one of the first and second housings may be rotated or otherwise agitated to facilitate the release of cells from the tissue sample and the flow of the cells through the outer porous wall 78 of the first housing.

According to this description, the apparatus may be used with numerous tissue sources where disaggregation is desired. For example, the apparatus may be used with adipose tissue or muscle which are among preferred sources of adult stem cells. The tissue-derived material that may be released includes cells, including individual cells, multi-cellular aggregates and cells associated with non-cellular material. The released cells may include more than one cell type. In some examples, the biological material also may be substantially non-cellular. In a preferred example, the tissue processor may be used to process adipose tissue to release stem cells.

In the example of adipose tissue, tissue may be obtained from a patient using conventional procedures including lipoaspiration or liposuction. The adipose tissue obtained from a patient may then be placed directly into the first housing or initially may be washed or otherwise treated before being placed in the first housing.

In one example, the tissue disaggregation process may involve the enzymatic treatment of the tissue sample. For example, collagenase digestion of connective tissue may be used to effect release of stem cells from adipose tissue. When enzymatic treatment is used, a solution of the enzyme may be added either directly to the first housing 76 where the tissue is located, or added to the space 94 between the walls of the first and second housings 76, 82 such that the enzyme diffuses from the inter-housing space 94 into the first housing 76.

After or during the disaggregation process, the flow of cells 90 from the first housing 76 through the porous outer wall 78 of the first housing 76 may be facilitated by flowing or pumping cell-compatible fluids through the first housing 76 such that cells are carried from the first housing through the porous outer wall 78 by the flow of the fluids. In one example, there may be a continuous flow of fluid through the first housing 76 to carry cells from the first housing through the porous outer wall 78 and to an outlet 86 located, for example, at the bottom of the second housing 82, as shown for example in FIG. 5.

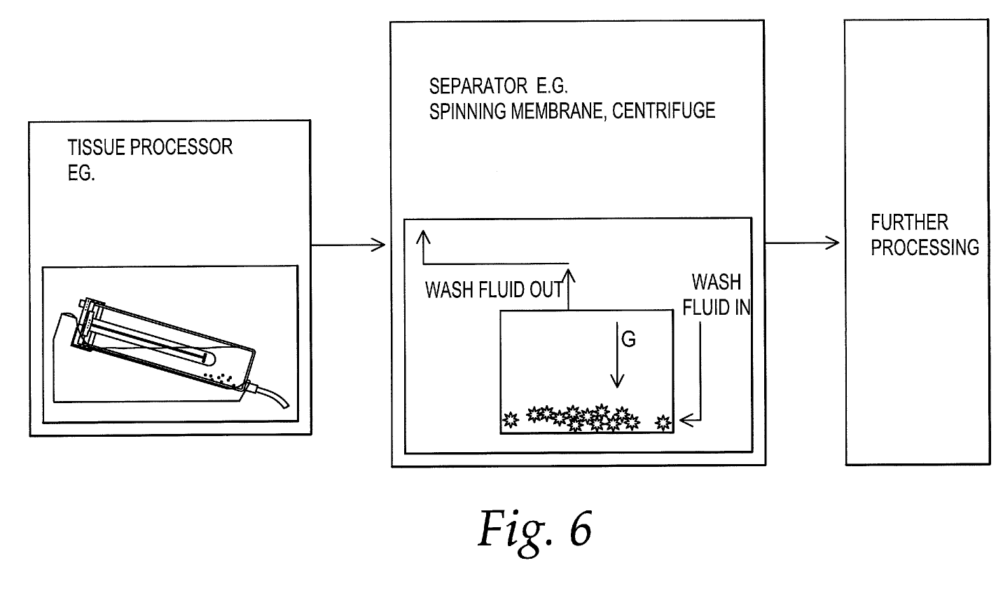

In one example, the apparatus may be directly linked to one or more systems or apparatus for further processing of materials. Tissue-derived material, including cells, may be flowed from the tissue processing apparatus through an outlet and may then flow to systems for washing, treating, purifying or further processing the cells. FIG. 6 is a schematic flow chart showing how the tissue processing apparatus may be part of systems for purifying cells. A pump (not shown), such as a peristaltic pump, may be included to facilitate the flow of material, such as cells, from the tissue processing apparatus to cell processing systems.

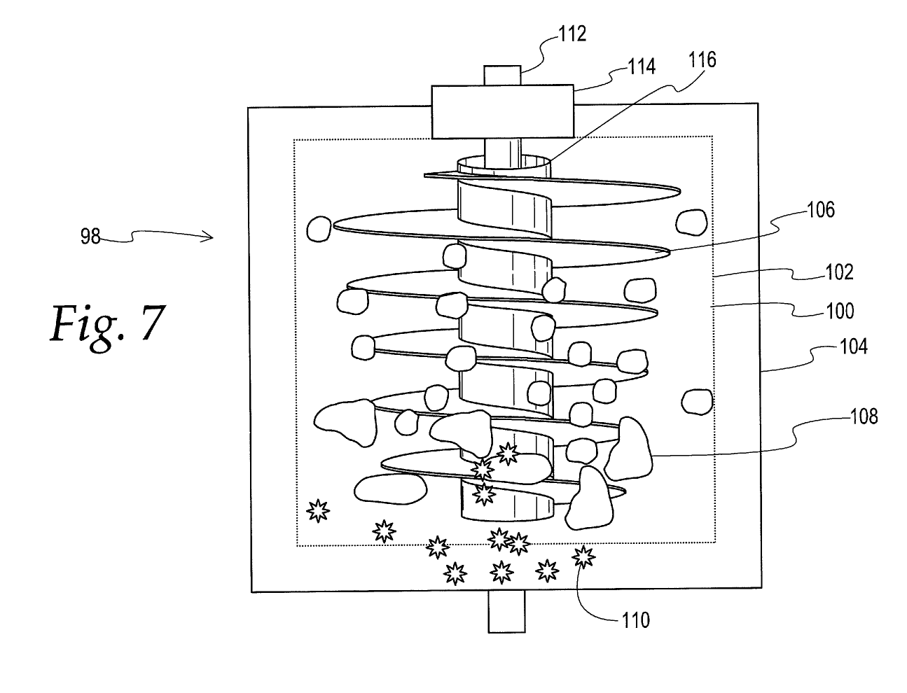

A further example of an apparatus for processing tissue 98 according to the disclosure is shown in FIG. 7. As in previous examples, the apparatus 98 includes a first housing 100 with a porous outer wall 102. The first housing 102 is adapted to receive a tissue sample and is enclosed within a second housing 104. According to this example, the apparatus also includes an agitator that is located within the first housing 100 to enhance tissue processing. The agitator may enhance tissue disaggregation by directly contacting the tissue to disassociate or tear the tissue, by creating shear effects within the first housing, by improving reagent and tissue mixing or by some combination of these effects.

In the example shown in FIG. 7, the agitator is an auger 106, although any other suitable configuration, such as paddle, beater or other may be used. The diameter and length of the auger as well as the pitch of the auger flighting may be selected according to particular requirements. An agitator, such as an auger, as described here may be used with any of the previously described examples of a tissue processing apparatus.

The apparatus 98 also preferably includes a drive mechanism for moving the agitator e.g. such as rotating an auger 106. In the example shown in FIG. 7, the auger is driven by motor 114 via drive shaft 112 mounted in a bearing 116. In other examples, a magnetic drive mechanism may be used to rotate the auger so that contents of the first and second housings may be completely sealed from the outside environment.

According to this example, as in previous examples, a tissue sample is placed within the first housing 100 which contains the auger 106. A solution containing a tissue disaggregation agent such as collagenase may be also placed within the first or second housings 100,104. During processing of the tissue sample, the auger 106 and the first housing 100 may both rotate relative to the second housing 104. The auger and first housing may rotate in the same or different directions and at the same or different speeds relative to each other. In a preferred example, the auger 106 and the first housing 100 rotate in different directions.

The direct contact of the auger 106 with tissue 108 may effect disassociation or tearing of the tissue into smaller fragments, enhancing tissue disaggregation. In addition, rotation of the auger may improve tissue disaggregation due to shear effects on the tissue and improved mixing of tissue and disaggregation reagents. As in previous examples, tissue disaggregation results in larger tissue fragments 108 being retained in the first housing 100 whereas cells 110 pass through the porous outer wall 102 of the first housing.

It will be understood that the examples of the present disclosure are illustrative of some of the applications of the principles of the present disclosure. Numerous modifications may be made by those skilled in the art without departing from the true spirit and scope of the disclosure. Various features which are described herein can be used in any combination and are not limited to particular combinations that are specifically described herein.

1-47. (canceled)

48. An apparatus for processing tissue to release cells from the tissue, comprising:
a first housing having a porous wall portion, the first housing adapted to receive a tissue sample and the wall portion being sufficiently porous to allow passage therethrough of material including cells derived from the tissue;
a second housing that at least substantially encloses the first housing;
at least one of the first and second housings being movable to assist in processing of tissue in the first housing and passage of material including cells derived from the tissue through the porous wall portion of the first housing; and
an agitator disposed within the first housing and movable relative to the first housing and second housing to enhance tissue processing therein.

49. The apparatus of claim 48 wherein the agitator includes a spiral agitation surface.

50. The apparatus of claim 48 in which the agitator is rotatable relatively to the first housing, the first housing has an axis and rotation of the agitator induces flow in an axial direction.

51. The apparatus of claim 50 in which rotation of the agitator also induces flow in a radial direction.

52. The apparatus of claim 48 wherein the agitator extends through substantially the full length of the first housing.

53. The apparatus of claim 48 wherein the agitator comprises an augur.

54. The apparatus of claim 48 wherein the agitator includes a drive shaft and the apparatus includes a motor external to the first housing operatively associated with the drive shaft.

55. The apparatus of claim 48 wherein the agitator includes a magnetic drive member.

56. The apparatus of claim 48 wherein the agitator is rotatable relative to the first housing and the first housing is movable through rotation and is rotatable in a direction opposite to the direction of rotation of the agitator.

57. The apparatus of claim 48 wherein the agitator is rotatable relative to the first housing and is configured such that upon rotation it creates shear forces on tissue within the first housing.

58. The apparatus of claim 48 wherein the agitator is configured for the processing of adipose tissue to release stem cells.

59. A method of using an apparatus for processing tissue to release cells from the tissue, comprising:
a. inserting a tissue sample containing cells into a first housing wherein the first housing has a porous wall portion, the first housing adapted to receive a tissue sample and the wall portion being sufficiently porous to allow passage therethrough of material including cells derived from the tissue, a second housing that at least substantially encloses the first housing and an agitator disposed within the first housing;
b. introducing at least one tissue-releasing agent into at least one of the housings; and
c. moving at least one of the first housing and the second housing and moving the agitator relative to the to process the tissue sample and to pass material including cells derived from the tissue sample through the porous outer wall of the first housing.

60. The method of claim 59 wherein the agitator includes a spiral agitation surface.

61. The method of claim 59 in which the agitator is rotatable relative to the first housing and the first housing has an axis and the rotation of the agitator induces flow in an axial direction.

62. The method of claim 61 in which rotation of the agitator also induces flow in a radial direction.

63. The method of claim 59 wherein the agitator comprises an auger.

64. The method of claim 59 wherein the agitator extends through substantially the full length of the first housing.

65. The method of claim 59 wherein the agitator includes a drive shaft and the apparatus includes a motor external to the first housing operatively associated with the drive shaft.

66. The method of claim 59 wherein the agitator is moved by a magnetic drive member.

67. The method of claim 59 wherein the agitator is rotatable relative to the first housing and the first housing is movable through rotation and is rotatable in a direction opposite to the direction of rotation of the agitator.

68. The method of claim 59 wherein the agitator is rotatable relative to the first housing and the agitator is configured such that upon rotation, it creates shear effects on tissue within the first housing.

69. The method of claim 59 wherein the tissue comprises adipose tissue.
